(19) United States
(12) Patent Application Publication
(10) Pub. No.: US 2024/0000856 A1
(43) Pub. Date: Jan. 4, 2024

(54) STABLE COMPOSITION COMPRISING A HONEY AND A CANNABINOID

(71) Applicant: MW PHARMA IP LIMITED, Auckland (NZ)

(72) Inventors: Mark James Dye, Whitianga (NZ); William Douglas, Auckland (NZ); Owen John Catchpole, Auckland (NZ); Teresa Moreno Rueda, Lower Hutt (NZ); Stephen John Tallon, Lower Hutt (NZ)

(21) Appl. No.: 18/039,392

(22) PCT Filed: Nov. 30, 2021
(86) PCT No.: PCT/NZ2021/050212

§ 371 Date: May 30, 2023

(30) Foreign Application Priority Data
Nov. 30, 2020 (NZ) 770492
Aug. 24, 2021 (AU) 2021221467

(51) Int. Cl. A61K 35/644; A61K 31/00; A61K 47/40; A61K 31/19

(52) U.S. Cl. CPC A61K 35/644; A61K 31/658; A61K 47/40; A61K 31/19

(57) ABSTRACT

Disclosed are compositions comprising a honey, a cannabinoid, and an emulsifier, and compositions comprising methylglyoxal, a cannabinoid and an emulsifier. The emulsifier is preferably cyclodextrin. The composition is stabilised such that the rate of degradation of the active components in honey, including methylglyoxal and dihydroxyacetone, is reduced. The composition is also physically stabilised such that the rate of separation of the cannabinoid is reduced and the homogeneity of the composition is maintained over time.

### FIELD

The present invention relates to a composition comprising a honey and a cannabinoid. More specifically, the present invention relates to a stabilised composition comprising Mānuka honey and a cannabinoid and methods for its preparation.

### BACKGROUND

Health and medical benefits of honey have been known for centuries. Honey has been a popular folk remedy for treatments of burns and other skin injuries, as a topical antibiotic, and for treatment of coughs and sore throats.

Mānuka honey is the honey produced by bees foraging on the Mānuka plant (Leptospermum scoparium).

Whilst antibacterial activity of honey has been ascribed to the presence of hydrogen peroxide, Mānuka honey has been shown to have additional antimicrobial activity not attributable solely to a single active ingredient. This additional activity is believed to be due, predominantly, to methylglyoxal (MGO) and dihydroxyacetone (DHA). DHA is a precursor compound that converts to MGO in honey over time and at a rate that increases with temperature.

The activity of Mānuka honey is often reported as a Unique Mānuka Factor (UMF) value. The UMF value correlates with the levels of MGO, and reflects the equivalent concentration of phenol (%, w/v) required to produce the same antibacterial activity as honey (Singh et al., AIMS Microbiol. 2018; 4(4): 655-664). Physiologically, MGO is regarded as a reactive oxygen species. The higher the UMF value, the higher the levels of activity of the Mānuka honey.

The activity of Mānuka honey, may also be reported by reference to its MGO level. Activity may also be reported as a Non-Peroxide Antibacterial Activity (NPA) (Cokcetin, Pappalardo, PLoS ONE, (2016) 11(12): e0167780).

Mānuka honey with a higher UMF or NPA value is more sought after by consumers looking for a honey with health benefits. High UMF value honey is also sought after for use in medical-grade honey products.

Other honeys, not necessarily considered to be Mānuka honey, are known to comprise MGO, including honeys produced from Australian Leptospermum species (Cokcetin, Pappalardo, PLoS ONE, (2016) 11(12): e0167780), and Kanuka honey, which is a honey derived from the flowers of Kunzea ericoides.

The presence, and level, of 5-hydroxymethylfurfural (HMF) is an indicator of the degradation of honey in general, and Mānuka honey in particular. HMF levels increase with the age of the honey, and increase when the honey is heated.

The UMF value of Mānuka honey decreases with increasing levels of HMF. UMF values of Mānuka honey also decrease when mixed with other ingredients. HMF levels tend to increase when mixed with other ingredients, especially where the added ingredients are chemically reactive.

Consequently, the shelf life of honey, and Mānuka honey in particular, is dependent on (1) its age, (2) whether it has been subjected to elevated temperatures, and/or (3) on the presence of chemically reactive additives.

Health and medical benefits of cannabinoids produced by the Cannabis plant are currently the subject of increased interest, in part due to the changing public attitudes towards the consumption of the compounds and easing of regulatory restrictions in respect of their use in several jurisdictions, including in the United States of America, United Kingdom, Europe, Canada, and New Zealand.

For example, cannabidiol (CBD) is believed to be effective in the treatment of neurological conditions including anxiety, pain, and movement disorders. Cannabigerol (CBG) is another cannabinoid with potential in antibacterial and anti-inflammatory applications.

Pure cannabidiol is a solid at room temperature. It is commercially available in several grades, including cannabidiol isolate, and as a broad spectrum oil. Cannabidiol isolate comprises cannabidiol which may be either solid or dissolved in a carrier oil. Broad spectrum oil is an oil extract from the Cannabis plant comprising predominantly cannabidiol, along with other cannabinoids.

As with other structurally similar cannabinoids, cannabidiol is known to degrade, at least in part via oxidation, over time, and by exposure to sunlight (see, for example, Moreno, T, Ind. Eng. Chem. Res. 2020, 59, 46, 20307-20315; R. Mechoulam, Chemistry and Physics of Lipids, 121, (2002), 35-43). The combination of cannabidiol with other chemically reactive compounds, such as reactive oxygen species, also increases the rate of its degradation.

There is a perception that both Mānuka honey and cannabinoids provide a natural alternative to synthetic or pharmaceutical remedies for the improvement of health and wellbeing, and/or the treatment of neurological or physical conditions and diseases. However, the combination of Mānuka honey and cannabinoids raises problems in the provision of a physically stable composition, at least in part because of the low solubility of cannabinoids in honey. Further, there are challenges in preparing a chemically stable composition in which the active components of both Mānuka honey and cannabinoids are protected from degradation.

### SUMMARY OF THE INVENTION

In an aspect, there is provided a composition comprising:
a honey;
a cannabinoid; and
an emulsifier.

In an embodiment, the composition consists essentially of a honey, a cannabinoid and an emulsifier.

Preferably, the honey comprises methylglyoxal. The honey may have a methylglyoxal content of ≥83 mg/kg, or ≥263 mg/kg, or ≥514 mg/kg.

Preferably, the honey comprises Mānuka honey. The Mānuka honey may have a UMF value of 5+. The Mānuka honey may have a UMF value of 10+, or of 15+ or greater.

Preferably, the water content of the composition is less than 20%.

Preferably, the composition comprises at least 0.5 mg cannabinoid per gram of composition. The composition may comprise between 0.5 mg and 15 mg cannabinoid per gram of composition.

Preferably, the cannabinoid is selected from tetrahydrocannabinol (THC), cannabidiol (CBD), cannabigerol (CBG), cannabinol (CBN), tetrahydrocannabinolic acid (THCA), cannabidiolic acid (CBDA), cannabichromene (CBC), tetrahydrocannabivarin, their salt, acid, and ester forms, or compounds that are structurally related to them. Cannabinoids that are structurally related to the aforementioned compounds comprise a six membered saturated, unsaturated, or aromatic (preferably unsaturated or aromatic) hydrocarbon ring bonded to: (1) hydroxyl groups or ether linkages at the 1- and 3-positions of the ring; (2) an alkyl chain (preferably C3-C8, more preferably C3-C5, alkyl chain) at the 5-position of the ring, relative the position of the groups at (1); and (3) an unsaturated alkyl chain at the 2-position of the ring (preferably a monoterpenoid moiety), relative to the position of the groups at (1), wherein the unsaturated alkyl chain may link to the ring via the ether linkage at (1).

For the purposes of the present application, cannabinoids include both plant-derived and non-plant derived cannabinoids.

In an embodiment, the cannabinoid comprises, or consists essentially of, cannabidiol. The composition may comprise at least 0.5 mg cannabidiol per gram of composition. The composition may comprise between 0.5 mg and 15 mg cannabidiol per gram of composition.

In another embodiment, the cannabinoid comprises, or consists essentially of, cannabigerol. The composition may comprise at least 0.5 mg cannabigerol per gram of composition. The composition may comprise between 0.5 mg and 15 mg cannabigerol per gram of composition.

In some embodiments, the composition preferably comprises at least 1 mg cannabinoid per gram of composition. In this embodiment, the composition preferably comprises at least 1 mg cannabinoid per gram of composition at 6 months after preparation, or at 12 months after preparation, or at 18 months after preparation, or at 24 months after preparation, particularly when stored at 25° C. or less.

In some embodiments, the composition preferably comprises at least 7 mg cannabinoid per gram of composition. In this embodiment, the composition preferably comprises at least 7 mg cannabinoid per gram of composition at 6 months after preparation, or at 12 months after preparation, or at 18 months after preparation, or at 24 months after preparation, particularly when stored at 25° C. or less.

In some embodiments, the composition comprises at least 1 mg cannabidiol per gram of composition. In this embodiment, the composition preferably comprises at least 1 mg cannabidiol per gram of composition at 6 months after preparation, or at 12 months after preparation, or at 18 months after preparation, or at 24 months after preparation, particularly when stored at 25° C. or less.

In other embodiments, the composition preferably comprises at least 7 mg cannabidiol per gram of composition. In this embodiment, the composition preferably comprises at least 7 mg cannabidiol per gram of composition at 6 months after preparation, or at 12 months after preparation, or at 18 months after preparation, or at 24 months after preparation, particularly when stored at 25° C. or less.

Preferably, the honey comprises dihydroxyacetone. The composition may comprise at least 250 mg dihydroxyacetone per kilogram of composition, and further comprise at least 250 mg dihydroxyacetone per kilogram of composition at 6 months after preparation, or at 12 months after preparation, or at 18 months after preparation, or at 24 months after preparation, particularly when stored at 25° C. or less.

The composition may comprise at least 250 mg methylglyoxal per kilogram of composition, and further preferably comprises at least 250 mg methylglyoxal per kilogram of composition at 6 months after preparation, or at 12 months after preparation, or at 18 months after preparation, or at 24 months after preparation, particularly when stored at 25° C. or less.

In some embodiments, the composition comprises no more than 45 mg 5-hydroxymethylfurfural per kilogram of composition, and further comprises no more than 45 mg 5-hydroxymethylfurfural per kilogram of composition at 6 months after preparation, or at 12 months after preparation, or at 18 months after preparation, or at 24 months after preparation, particularly when stored at 25° C. or less.

In some embodiments, the emulsifier may comprise, or may consist essentially of, lecithin. In these embodiments, the ratio of lecithin:cannabinoid is at least 0.5:1 (w/w). Preferably, the ratio of lecithin to the cannabinoid is at least 2:1 (w/w), for example, the ratio of lecithin to the cannabinoid may be between 2:1 and 10:1 (w/w).

In some embodiments, the emulsifier may comprise, or may consist essentially of, cyclodextrin. The cyclodextrin may include alpha-, beta-, gamma-cyclodextrin, or combinations thereof. In these embodiments, the ratio of cyclodextrin to cannabinoid is preferably at least 3:1 (w/w). Preferably, the ratio of cyclodextrin to cannabinoid is greater than 4:1 (w/w). In one preferred example, the ratio of cyclodextrin to cannabinoid is about 9:1 (w/w). The molar ratio of cyclodextrin:cannabinoid is preferably at least 1:1. Preferably, the molar ratio of cyclodextrin:cannabinoid is preferably at least 2:1. In these embodiments, the composition preferably comprises a cyclodextrin-cannabinoid complex. The complex may comprise stabilising interactions, such as non-covalent bonds or hydrogen bonds, between cyclodextrin and cannabinoid.

Preferably, the composition comprises at least 90% w/w honey.

Preferably, the cannabinoid is uniformly distributed in the composition, and preferably remains uniformly distributed at 6 months after preparation, or at 12 months after preparation, or at 18 months after preparation, or at 24 months after preparation.

In another aspect, there is provided a method for preparing the composition of the present invention, comprising steps of:
a) Combining the cannabinoid and the emulsifier to form a cannabinoid-emulsifier combination; and
b) Combining the combination with the honey.

In some embodiments, the emulsifier comprises, or consists essentially of, lecithin. In these embodiments, the cannabinoid and lecithin are preferably combined at a temperature of less than 50 degrees C.

In some embodiments, the emulsifier comprises, or consists essentially of, cyclodextrin, for example, alpha-cyclodextrin, beta-cyclodextrin, gamma-cyclodextrin, and combinations thereof. In these embodiments, the cyclodextrin and cannabinoid are combined in the presence of water and a non-aqueous solvent. In these embodiments, the cannabinoid and cyclodextrin are preferably combined at a temperature of less than 50 degrees C. The solvent preferably comprises a water-miscible solvent, such as isopropyl alcohol. The cyclodextrin-cannabinoid complex is preferably dried prior to step b. Preferably, the cannabinoid-emulsifier combination is provided as a powder prior to step b.

In another aspect, there is provided a method for extending the shelf life, or length of time of substantial uniformity, of a composition comprising a cannabinoid and a honey, comprising a step of combining the cannabinoid and an emulsifier to form a cannabinoid-emulsifier combination.

In another aspect, there is provided a composition comprising:
Methylglyoxal;
A cannabinoid; and
An emulsifier.

Preferably, the methylglyoxal is derived from honey. Preferably, the composition further comprises a honey.

### Definitions

Broad spectrum oil (BSO): An oil extract from the Cannabis plant (such as Cannabis sativa and Cannabis indica) comprising several cannabinoids, predominantly cannabidiol. Broad spectrum oil may be diluted in a carrier oil, such medium chain triglyceride oil.

Cannabinoid: A compound structurally related to tetrahydrocannabinol and cannabidiol. Cannabinoids include tetrahydrocannabinol (THC), cannabidiol (CBD), cannabigerol (CBG), cannabinol (CBN), tetrahydrocannabinolic acid (THCA), cannabidiolic acid (CBDA), cannabichromene (CBC), and tetrahydrocannabivarin, their salt, acid, and ester forms. For the purposes of the present application, cannabinoids include both plant-derived and non-plant derived cannabinoids.

Cannabidiol: A cannabinoid having the IUPAC chemical formula 2-[(1R,6R)-6-isopropenyl-3-methylcyclohex-2-en-1-yl]-5-pentylbenzene-1,3-diol, and the structure shown below:

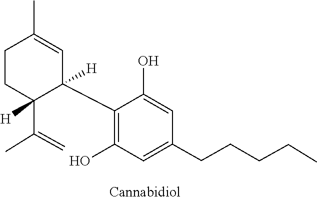 [SMILES: C=C(C)[C@@H]1CCC(C)=C[C@H]1c1c(O)cc(CCCCC)cc1O]

Cannabidiol isolate: Substantially purified cannabidiol. Cannabidiol isolates typically comprise at least 98% w/w cannabidiol.

Cannabigerol: A cannabinoid having the IUPAC chemical formula 2-[(2E)-3,7-dimethylocta-2,6-dienyl]-5-pentyl-benzene-1,3-diol, and the structure shown below:

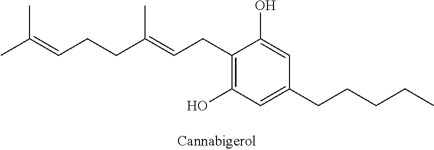 [SMILES: CCCCCc1cc(O)c(C/C=C(\C)CCC=C(C)C)c(O)c1]

Mānuka honey: Monofloral honey produced by bees who harvest the nectar of Leptospermum scoparium.

UMF: Unique Mānuka Factor: A measurement of the antibacterial activity of honey, based on the equivalent concentration of phenol (% w/v) required to produce the same antibacterial activity as honey. UMF values are primarily based on the measured level of MGO such that UMF 5+ honey has ≥83 mg/kg MGO, UMF 10+ has ≥263 mg/kg MGO, and UMF 15+ has ≥514 mg/kg MGO (Unique Mānuka Factor Honey Association. Grading System Explained 2019. https://www.umf.org.nz/grading-system-explained/. [last accessed 22 Nov. 2021]).

### DETAILED DESCRIPTION

The present invention provides stable compositions comprising honey and a cannabinoid. More specifically, the present invention provides stable compositions comprising Mānuka honey, a cannabinoid, and an emulsifier. In addition, the present invention provides stable compositions comprising MGO, a cannabinoid, and an emulsifier. The present invention is predicated on the surprising finding that the combination of a cannabinoid and an emulsifier, cyclodextrin in particular, reduces the rate of degradation of the cannabinoid by active components of honey, and reduces the rate of degradation of active components of honey by cannabinoids.

### Honey

Honey comprising MGO is preferred. Honey comprising MGO and DHA is more preferable. Preferred honeys comprise a methylglyoxal content of ≥83 mg MGO/kg honey, or ≥263 mg MGO/kg honey, or ≥514 mg MGO/kg honey. Accordingly, Mānuka honey (which naturally comprises MGO and DHA) is an example of a preferred honey. In preferred examples, Mānuka honey has a UMF value of at least 5 (5+), at least 10 (10+) or at least 15 (15+). Mānuka honey is preferred due to the high levels of active components conferring a health benefit and/or consumer appeal, including hydrogen peroxide, MGO, and DHA. Typical water content of Mānuka honey is about 17-19% w/w.

Alternative preferred honeys include honeys which may not be considered to be a Mānuka honey or a monofloral honey from Leptospermum scoparium, but nonetheless comprise MGO and/or DHA. Examples include honey derived from other Leptospermum species, and Kanuka honey, which is a honey derived from the flowers of Kunzea ericoides.

### Cannabinoid

Any cannabinoid is suitable for the compositions of the present invention. As shown herein, cannabinoids may be effectively combined or complexed with an emulsifier in order to reduce the reactivity of the cannabinoid with active components of honey, and to improve stability and uniformity in the composition.

Experiments described herein show the stabilisation of compositions comprising a cannabinoid and a honey in which the cannabinoid is cannabidiol (CBD) or cannabigerol (CBG). Structurally, CBD and CBG share a common 5-pentyl-benzene-1,3-diol moiety. CBD and CBG differ in aspects of their respective monoterpenoid moieties at the 2-position on the benzene ring, although both CBD and CBG comprise similar alkene functionality in this moiety. CBD and CBG are therefore representative of a wider class of structurally related cannabinoids which generally share a six membered saturated or unsaturated hydrocarbon ring, an alkyl chain at the 5-position of the ring, either hydroxyl groups or oxygen linkages (or ether groups) at the 1- and 3-positions, and an unsaturated alkyl chain at the 2-position (typically a terpenoid, or monoterpenoid, moiety). Cannabinoid compounds sharing this moiety are expected to show similar physical and chemical stability compared to the CBD and CBG compositions exemplified herein, due to them having similar solubilities in honey, similar affinities for emulsifiers (such as cyclodextrin) and similar functional groups. Particularly preferred cannabinoids sharing these features include tetrahydrocannabinol (THC), cannabidiol (CBD), cannabigerol (CBG), cannabinol (CBN), tetrahydrocannabinolic acid (THCA), cannabidiolic acid (CBDA), cannabichromene (CBC), and tetrahydrocannabivarin. Preferably, the cannabinoid includes the salts, acid and ester forms of these compounds.

Cannabidiol (CBD) is a preferred cannabinoid due to its health benefits and/or consumer appeal in assisting with conditions including anxiety, pain and movement disorders. Cannabigerol (CBG) is a further preferred cannabinoid due to its potential in antibacterial, anti-inflammatory, and other health applications.

The cannabinoid may be provided as a pure compound or as an extract. For example, cannabidiol may be provided as a pure compound, such as cannabidiol isolate, or as an extract, such as broad spectrum oil. Cannabidiol levels in broad spectrum oil are variable depending on supplier, but are typically between about 30% and 50%. Similarly, cannabigerol may be provided as a pure compound or as an extract.

Preferred compositions of the present invention comprise between about 0.5 and 10 mg cannabinoid per gram of composition or between about 0.5 and 15 mg cannabinoid per gram of composition. Examples 1 to 5 describe compositions comprising cannabinoid levels of approximately 1 mg/g and 7 mg/g.

### Emulsifier

Cannabinoids comprise a number of functional groups which are reactive with active components of Mānuka honey, such as hydrogen peroxide, MGO and DHA. Therefore, being able to provide a composition comprising both Mānuka honey and a cannabinoid requires a reduction of the reactivity between the active components of honey and the cannabinoid.

Active components in Mānuka honey, such as MGO and DHA and hydrogen peroxide, are prone to degradation by reaction with additives to the honey. For example, MGO and DHA are believed to be prone to reaction with, and degradation by, cannabinoids. The inventors believe that the combination of a cannabinoid and emulsifier to produce a cannabinoid-emulsifier combination reduces the rate of degradation of the active components in compositions of the present invention. Further, association of the cannabinoid with the emulsifier is believed to protect the cannabinoid from degradation (e.g., oxidative degradation) by the active components of honey, and MGO in particular.

Cyclodextrin is one preferred emulsifier. Gamma cyclodextrin (γ-CD) is particularly preferred. Combination of cyclodextrin with a cannabinoid according to the method described herein produces a cyclodextrin-cannabinoid complex. The complex comprises stabilising interactions, such as non-covalent bonds and/or hydrogen bonds, between the cannabinoid and cyclodextrin.

Without wishing to be bound by theory, it is believed that the cannabinoid interacts with the interior of the cyclodextrin ring. Stabilising interactions between the functional groups of cyclodextrin and the cannabinoid are believed to retain the cannabinoid, at least partially, in the interior of the cyclodextrin ring. As a result of the interactions between the cannabinoid and cyclodextrin, the cyclodextrin-cannabinoid complex is more soluble in a honey composition compared to non-complexed cannabinoid.

In embodiments of the invention comprising cyclodextrin as the emulsifier, the composition preferably comprises cyclodextrin:cannabinoid in a molar ratio of at least 1:1 such that, at the molecular level, each cannabinoid molecule is complexed with a molecule of cyclodextrin. To account for less than perfect complexation efficiency during production, and/or for the complexation of more than one cyclodextrin molecule with a cannabinoid molecule, the molar ratio is preferably greater than 1:1 cyclodextrin:cannabinoid.

Analysis of cannabinoid content in the tops and bottoms of post-centrifuged samples in Example 3 indicated that uniformity of cannabinoid in the composition were best retained when the ratio of cyclodextrin to cannabinoid was greater than 4:1 (w/w). Best results were observed when the ratio of cyclodextrin to cannabinoid was 9:1 (w/w).

From a perspective of chemical and physical stability of the composition, the inventors believe that there is no upper limit on the amount of cyclodextrin in the composition of the present invention.

Without wishing to be bound by theory, it is believed that reduction in the rate of degradation of cannabinoid in the honey compositions is due to the complexation of the cannabinoid with the emulsifier. In particular, it is believed that the complexation of cannabinoid with the emulsifier reduces the rate of degradation of cannabinoid by reaction with Mānuka honey active components, such as MGO and DHA. Conversely, it is believed that the reduction in the rate of degradation of active components of honey (e.g., MGO and DHA) by the cannabinoid is due to the complexation of cannabinoid with the emulsifier.

Consequently, it is believed that the use of cyclodextrin in complexing with cannabinoid increases the stability of a composition comprising a honey and a cannabinoid, and therefore extends the shelf life of the composition.

Lecithin is an alternative emulsifier. The major components of lecithin are the phosphatidylcholine class of phospholipids, and it is believed that these components contribute to the stabilisation of cannabinoids in the compositions of the present invention in which lecithin is the emulsifier. Whilst sunflower lecithin was used in the compositions described in the Examples (see below), any source of lecithin would be suitable for compositions of the present invention. Ideally, the lecithin used is a liquid at normal operational temperatures.

In embodiments of the invention comprising lecithin as the emulsifier, the composition preferably comprises lecithin:cannabinoid in a weight ratio of at least 0.5:1, such that the cannabinoid is sufficiently stabilised in the composition. From a perspective of chemical and physical stability of the composition, the inventors believe that there is no upper limit on the amount of lecithin in the composition of the present invention.

Without wishing to be bound by theory, it is believed that lecithin stabilises domains of an oil phase comprising cannabidiol in the honey/cannabidiol compositions. Domains of cannabidiol-containing oil suspended in honey are stabilised by lecithin. This interaction reduces the rate of coalescence of oil domains in the honey, which leads to a lower rate of separation of the honey and oil phases, and extends the length of time in which the composition is homogeneous.

### Composition

In preferred compositions, the honey and the cannabinoid are present in relative quantities such that a serving of the composition (which is about one teaspoon, or about 7 grams) provides the consumer with a benefit from both the honey component and cannabinoid component.

The preferred amount of cannabinoid may vary depending on the therapeutic dosage recommendations for each cannabinoid. The upper and lower limits of cannabinoid in the composition of the present invention are related to its desired therapeutic effect, and therefore related to the typical serving size of the composition. However, for most cannabinoids, a preferred range is about 0.5 to about 10 mg, or about 0.5 to about 15 mg, cannabinoid per gram of honey.

Where the composition comprises cannabidiol, preferred compositions comprise about to 10 mg cannabidiol per gram of honey. The range of 0.5 to 10 mg/g is considered to provide the desired therapeutic effect in a serving of about 7 g of composition.

Where the composition comprises cannabigerol, preferred compositions comprise about 0.5 to 10 mg cannabigerol per gram of honey. The range of 0.5 to 10 mg/g is considered to provide the desired therapeutic effect in a serving of about 7 g of composition.

The compositions of the present invention preferably have a water content that is close to the water content of natural honey. A benefit of the present invention is that no additional water is required for the combination of honey, an emulsifier and a cannabinoid. Accordingly, the composition preferably has a water content of 20% w/w or less.

A further consideration is that the emulsifier and cannabinoid interact to form a stabilised cannabinoid-emulsifier system, such that the reactivity of cannabinoid with active components of honey is reduced. Where the emulsifier is a complex-forming emulsifier such as cyclodextrin, the stabilised cannabinoid-emulsifier system is a cyclodextrin-cannabinoid complex. Where the emulsifier is a micelle-forming emulsifier such as lecithin, the stabilised cannabinoid-emulsifier system is a cannabinoid micelle stabilised by lecithin.

### Mouthfeel and Other Organoleptic Properties

It has been found that the presence and type of the emulsifier affects the mouthfeel and other organoleptic properties of the composition of the present invention.

The emulsifier modulates the viscosity of the composition, which affects mouthfeel. For compositions comprising Mānuka honey and cyclodextrin-cannabinoid complexes described herein, the viscosity of the composition is substantially the same as the viscosity of Mānuka honey.

The formation of cannabinoid-emulsifier systems, for example with cyclodextrin, affects the taste, smell, and flavour of the composition. Compositions comprising cyclodextrin have been found to have a reduced flavour of cannabinoid components. This is believed to be due to the complexation of the cannabinoid with cyclodextrin.

It has further been found that the compositions comprising Mānuka honey and the cyclodextrin-cannabinoid complexes described herein have a complementary texture profile, even for compositions with a 9:1 (w/w) ratio of cyclodextrin:cannabinoid.

Physical Stability/Tendency to Separate

It is critical for compositions comprising cannabinoids to have a physically stable, uniform dispersion of cannabinoid throughout the composition, and to remain uniform for their whole shelf life. To this end, it is advantageous for a composition to retain a uniform dispersion of cannabinoid for as long as possible in order to extend its shelf life. However, cannabinoids dispersed through other media, such as honey, have a tendency to segregate and form regions of higher and lower concentrations of cannabinoid in the composition. It has been found that segregation of cannabinoids in honey can be avoided for an extended period of time with an emulsifier-cannabinoid combination.

Preferred compositions are physically stable such that the components of the composition, especially cannabinoids, are uniformly dispersed throughout the composition for a commercially useful period of time. Preferably, the period of such physical stability and uniformity of distribution of cannabinoid is at least six months, more preferably at least 12 months, more preferably at least 18 months and most preferably at least 24 months.

Formulation 1 in Example 1 (Mānuka honey, broad spectrum oil, and lecithin) was shown to retain a uniformly dispersed level of cannabidiol throughout the composition after centrifugation at 2,400 rpm for ten minutes.

Formulation 2 (Mānuka honey, broad spectrum oil, and cyclodextrin) in Example 1 did not comprise an even distribution of cannabidiol after centrifugation. The physical instability of Formulation 2 is due to the incomplete complexation of the cannabinoid with cyclodextrin. Example 2 shows that successful complexation of cannabidiol with cyclodextrin does in fact provide an even distribution of cannabidiol after centrifugation. Example 2 further shows that whilst complexation of cannabidiol with cyclodextrin is critical to a uniform distribution of cannabidiol after centrifugation, the dissolution of the cyclodextrin-cannabidiol complex into honey is also critical.

As shown in the Examples and accompanying Tables, the processing temperatures used for mixing with the honey did not appear to have any effect on the cannabidiol properties over the range tested.

A formulation comprising broad spectrum oil and Mānuka honey without any emulsifier gave the strongest indications of segregation with a concentration of 0.7 mg/g in the bottom fraction and 2.9-3.6 mg/g in the top fraction (see Tables 1-3). A preformulated broad spectrum oil in medium chain triglyceride oil, when mixed with Mānuka honey in the absence of any added emulsifier, showed a moderate susceptibility to segregation under centrifugation (See Tables 1-3).

Formulations in clover honey gave substantially similar cannabidiol concentration results to their Mānuka honey counterparts.

Examples 1˜4 describe the centrifugation of sample tubes containing compositions of the present invention at 2400 rpm for 10 minutes, followed by a comparison of the levels of cannabinoid at the top and bottom of each sample. The results show that the compositions remained substantially uniform in concentration between the tops and bottoms. The centrifugation stability test described herein is considered to be an extreme test of physical stability. Evidence of separation under these conditions is not a direct indication that separation will occur in the same way under normal gravity and storage conditions. However, it is a good indicator or relative stability and relative tendency to separate. Therefore, the fact that the compositions of the present invention remained substantially homogeneous after centrifugation, particularly the compositions comprising honey and cyclodextrin-cannabinoid complexes, indicates good physical stability and homogeneity over time, e.g., at least six months, more preferably at least 12 months, more preferably at least 18 months and most preferably at least 24 months, particularly when stored at 25° C. or less.

### Chemical Stability of Mānuka Honey Actives and Cannabinoids

MGO and DHA levels in Mānuka honey were substantially unaffected by the processing steps to prepare the compositions of the present invention. Analysis of the Mānuka honey starting material and the compositions of the present invention showed that the levels of MGO, DHA and HMF levels were substantially similar.

Cannabinoid levels were substantially unaffected by the processing steps in the preparation of the compositions of the present invention.

Example 5 describes analysis of the stability of cannabinoid and active components of Mānuka honey in compositions stabilised with either lecithin or cyclodextrin. The compositions were subjected to accelerated aging conditions (40° C. and 75% RH) as well as standard conditions (25° C. and 60% RH), in order to determine likely stability of the compositions over periods of 12, 18 or 24 months. CBD is known to convert into degradation products over time in a reaction that is accelerated by increasing temperature (Moreno, T., et al. (2020). “Cannabinoid Decarboxylation: A Comparative Kinetic Study.” Industrial & Engineering Chemistry Research 59(46): 20307-20315), and therefore observed changes obtained from samples subjected to higher temperatures is indicative of what would be expected over a longer duration under standard conditions.

The rates of degradation of the cannabinoid and Mānuka honey active components were significantly reduced under both standard conditions as well as accelerated aging conditions in compositions comprising cyclodextrin compared to compositions comprising lecithin.

The rate of degradation of active components of honey, including MGO and DHA, as well as cannabinoids, increases with temperature. Shelf life of the composition is therefore improved by avoiding high temperatures during storage. As shown in Example 5, the MGO content in cyclodextrin-containing compositions stored at room temperature was substantially the same as observed for the control. For example, MGO levels were unchanged over 6 months of storage at 25° C., and DHA levels remained above 250 mg/kg after 6 months of storage at 25° C. The cannabinoid content in cyclodextrin-containing compositions was substantially constant over the 6 month period at 25° C., although cannabinoid content in lecithin-containing compositions underwent some decrease.

### Method of Preparation

The compositions of the invention may be prepared by admixing a cannabinoid with an emulsifier and a honey.

In an example, the compositions are prepared by a first step of admixing a cannabinoid with an emulsifier to produce a cannabinoid/emulsifier combination, and a subsequent step of admixing the cannabinoid/emulsifier combination with honey. Where the emulsifier is cyclodextrin, the step of producing a cannabinoid/emulsifier combination comprises the combination of cannabinoid and cyclodextrin to produce a cyclodextrin-cannabinoid complex. A preferred method for preparing cyclodextrin-cannabinoid complexes comprises the use of a water miscible solvent such as isopropyl alcohol to assist the complexing process. Alternative water miscible solvents suitable for the present invention include solvents suitable for use in the manufacture of food products, such as ethanol. Following the preparation of the cyclodextrin-cannabinoid complex, there is an evaporation step to dry the complex. This may be achieved with freeze drying or spray drying. As described in Example 2, the cyclodextrin-containing compositions had a low aroma and light colour, which is indicative of good complex formation.

In preferred methods of preparation, the step of admixing the cannabinoid-emulsifier combination with honey is performed without the incidental incorporation, or at least excessive incorporation, of gases. Alternatively, an optional step of degasification may be performed after the admixing of the cannabinoid/emulsifier combination with honey. It is believed that degasification (e.g., deaeration) of the composition, which occurs over time, may increase the rate of phase separation, as the gases rise through the composition. Therefore, an active step of degasification may be performed to avoid or reduce natural degasification over time.

In an example, the method of preparation of the compositions of the present invention comprises the steps of: (1) combining an aqueous solution of cyclodextrin with a cannabinoid dissolved in a water miscible solvent, to produce a solution comprising a cyclodextrin-cannabinoid complex; (2) optionally, adding water or water miscible solvent to the solution of cyclodextrin-cannabinoid complex; (3) drying the solution of cyclodextrin-cannabinoid by spray drying (optionally at a temperature of 50° C. or less) or by evaporation (optionally at a temperature of 50° C. or less) followed by freeze drying to produce a dried cyclodextrin-cannabinoid complex; and (4) combining the cyclodextrin-cannabinoid complex with honey.

### EXAMPLES

### Materials

Broad spectrum oil containing 33% cannabidiol was obtained from Dragonfly Biosciences, United Kingdom; Full spectrum distillate (BSD) containing 72% cannabidiol was obtained from Casco Bay Hemp, USA; Cannabidiol in MCT oil (10% cannabidiol) was obtained from Dragonfly extracts; Cannabidiol isolate 99% was obtained from Casco Bay Hemp, USA; Cannabigerol isolate 99.6% was obtained from Sanobiotec Novus UAS; Mānuka honey (UMF was obtained from ORAF Foods (New Zealand). Clover honey was obtained from Arataki Honey, New Zealand; Sunflower lecithin in liquid form was obtained from Soya International; Gamma-cyclodextrin (Cavamax W8) was obtained from Wacker.

The Mānuka honey was analysed prior to use, and found to have a DHA:MGO ratio of 1.2, and a HMF level of about 35 mg/kg.

### Example 1

Formulation 1—Composition Comprising Broad Spectrum Cannabidiol Oil, Mānuka Honey and Lecithin

Broad spectrum oil (1 g) was mixed with liquid lecithin (9 g) and warmed to approx. 45° C. with stirring until the mixture was well blended. 3 g of the blend was then added to 197 g of honey that had been prewarmed to approx. 50° C. The mixture combined easily and was stirred for a few minutes with no visible separation.

Formulation 2—Composition Comprising Broad Spectrum Cannabidiol Oil, Mānuka Honey and Cyclodextrin

Gamma-cyclodextrin (γ-CD) was mixed with water at a 1:1 mass ratio (80 g γ-CD+80 g water) to produce a paste, ensuring that all solids were uniformly mixed in. 47 g of a concentrated solution (50%) of broad spectrum oil in isopropyl alcohol (IPA) was added to the γ-CD paste under continuous stirring until blended. Additional water (250 mL) was added under continuous stirring to ensure the final mixture contained less than 5% IPA, resulting in a low viscosity aqueous suspension. The suspension was then freeze-dried.

1.22 g of the freeze-dried powder was finely ground and admixed to approx. 199 g of honey that had been prewarmed to approx. 50° C.

For Formulations 1 and 2, the levels of MGO and DHA were measured throughout the formulation process and were observed to be stable.

Stability

Samples of warm Formulations 1 and 2 were transferred to centrifuge tubes and centrifuged for 10 minutes at 2,400 rpm. Subsamples from the top and bottom of the tubes were collected. Formulation 1 appeared visually uniform, and analysis of the top and bottom subsamples showed uniform amounts of cannabidiol. Formulation 2 showed a foamy layer at the top of the tube, and analysis of the top and bottom subsamples showed higher concentrations of cannabidiol in the top subsamples compared to the bottom subsamples, suggesting that some poorly mixed cannabidiol complex had separated to the top surface. The cyclodextrin complex appeared granular suggesting agglomeration and non-uniformity.

### Example 2

Formulations 3-5—Composition Comprising Broad Spectrum Cannabidiol Oil, Mānuka Honey and Lecithin

Stock mixtures of broad spectrum oil (BSO) in lecithin were prepared by combining the required ratios and then warming to approx. 45° C. with stirring until the mixture was well blended. The BSO comprised 33% cannabidiol by weight. Formulations 3 to 5 had the following BSO:lecithin (w/w) ratios:
Formulation 3: 1:10 (33 g total)
Formulation 4: 1:5 (6 g total)
Formulation 5: 1:2 (3 g total)

Subsamples of the stock solution were then mixed with honey by warming the honey to a target temperature (50, 60, or 70° C.), adding the required amount of lecithin-BSO mixture to give an overall cannabidiol concentration of 0.14 wt. % or 0.70 wt. % (i.e., 1.4 mg/g or 7 mg/g), and stirring for 30 minutes.

Formulation 7—Composition Comprising Cannabidiol Isolate and Lecithin

A mixture of cannabidiol isolate powder and lecithin (1:10 cannabidiol isolate:lecithin ratio, w/w, equivalent to 1:10 cannabidiol:lecithin w/w) was prepared by stirring at approximately 45° C. until dissolved. The mixtures blended well with no visible non-uniformity after mixing.

The cannabidiol-lecithin combination was then mixed with honey at different target ratios and temperatures. Examples of compositions, and the analysis thereof, are shown in Tables 1-3.

Formulation 8—Composition Comprising Broad Spectrum Oil, Mānuka Honey and Cyclodextrin

γ-CD was mixed with water at a 1:1 mass ratio (18 g γ-CD+18 g water) to produce a uniform paste. A solution of broad spectrum oil (2 g) in isopropyl alcohol (IPA, 6 g) was prepared separately. The ratio of IPA to broad spectrum oil used was higher than in the experiment described in Example 1, with the aim of improving the broad spectrum oil mobility in the mixture to aid the cannabidiol-cyclodextrin complex formation.

The broad spectrum oil solution was added to the γ-CD solution while mixing. Additional water (40 g) was added during mixing to achieve a uniform mixture. The mixture was partially evaporated under vacuum at <35° C. and then freeze dried. The freeze-dried powder was ground to a fine powder. 18.6 g of complex was produced. The powder was a light brown colour without noticeable aroma.

The γ-CD complex powder was then mixed with honey in different ratios and temperatures. Examples of compositions, and the analysis thereof, are shown in Tables 1-3.

Formulation 9—Composition Comprising Cannabidiol Isolate, Mānuka Honey and Cyclodextrin

A complex powder using γ-CD and cannabidiol isolate was prepared in a similar manner to Formulation 8. Cannabidiol isolate powder (2 g) was dissolved into IPA (6 g), and admixed with γ-CD in water. After evaporation, freeze drying, and grinding, 18.6 g of white aroma-free powder was produced.

The γ-CD complex powder was then mixed with honey in different ratios and temperatures. Examples of compositions, and the analysis thereof, are shown in Tables 1-3.

TABLE 1
Cannabidiol content in the honey/cannabidiol/emulsifier (carrier) compositions.

| | | | | | CBD in | | |
|---|---|---|---|---|---|---|---|
| | Mix Temp | CBD | | Carrier:CBD | honey | | |
| Honey* | (° C.) | source | Carrier | source (wt:wt) | (mg/g) | Centrifuged | Fraction # |
| M | 50 | BSO | Lec | 10:1 | 1.4 | | 1 |
| M | 60 | BSO | Lec | 10:1 | 1.4 | | 4 |
| M | 70 | BSO | Lec | 10:1 | 1.4 | | 5 |
| M | 50 | BSO | Lec | 10:1 | 7.0 | Y | 2 |
| C | 50 | BSO | Lec | 10:1 | 1.4 | | 3 |
| M | 50 | BSO | Lec | 5:1 | 1.4 | | 6 |
| M | 50 | BSO | Lec | 2:1 | 1.4 | | 7 |
| M | 50 | Iso | Lec | 10:1 | 1.4 | | 8 |
| M | 50 | Iso | Lec | 10:1 | 7.0 | Y | 9 |
| M | 50 | BSO | CD | 9:1 | 1.4 | Y | 14 |
| M | 60 | BSO | CD | 9:1 | 1.4 | | 19 |
| M | 70 | BSO | CD | 9:1 | 1.4 | | 20 |
| C | 50 | BSO | CD | 9:1 | 1.4 | | 17 |
| M | 50 | Iso | CD | 9:1 | 1.4 | Y | 21 |
| M | 50 | BSO | — | — | 1.4 | Y | 13 |
| M | 50 | BSO | MCT | 3:1** | 1.3 | Y | 12 |
| M | 50 | BSO | CD | 3.4:1 | 1.6 | Y | 18 |

*M = Manuka Honey; C = Clover Honey; BSO = Broad Spectrum Oil; Iso = CBD Isolate.
**Premixed by supplier to give ~10% CBD concentration.

TABLE 2
Cannabidiol content of honey/cannabidiol/emulsifier (carrier) compositions

| | | | | Carrier:CBD | CBD in | | | | | | | |
|---|---|---|---|---|---|---|---|---|---|---|---|---|
| | Mix | CBD | | source | honey | | CBD content (mg/g) | | | | | |
| Honey | Temp | source | Carrier | (wt:wt) | (mg/g) | Fraction | Bulk | | Top | | Bottom | |
| M | 50 | BSO | Lec | 10:01 | 1.4 | 1 | 1.8 | 1.6 | | | | |
| M | 60 | BSO | Lec | 10:01 | 1.4 | 4 | 1.8 | 1.7 | | | | |
| M | 70 | BSO | Lec | 10:01 | 1.4 | 5 | 1.9 | 1.8 | | | | |
| M | 50 | BSO | Lec | 10:01 | 7 | 2 | 7.5 | | 7.5 | | 6.5 | |
| C | 50 | BSO | Lec | 10:01 | 1.4 | 3 | 1.8 | 1.8 | | | | |
| M | 50 | BSO | Lec | 5:01 | 1.4 | 6 | 1.5 | 1.4 | | | | |
| M | 50 | BSO | Lec | 2:01 | 1.4 | 7 | 1.4 | 1.4 | | | | |
| M | 50 | Iso | Lec | 10:01 | 1.4 | 8 | 1.5 | 1.5 | | | | |
| M | 50 | Iso | Lec | 10:01 | 7 | 9 | 7 | | 8.1 | | 7.5 | |
| M | 50 | BSO | CD | 9:01 | 1.4 | 14 | 1.4 | 1.3 | 1.4 | 1.3 | 1.3 | 1.3 |
| M | 60 | BSO | CD | 9:01 | 1.4 | 19 | 1.4 | 1.3 | | | | |
| M | 70 | BSO | CD | 9:01 | 1.4 | 20 | 1.3 | 1.3 | | | | |
| C | 50 | BSO | CD | 9:01 | 1.4 | 17 | 1.4 | 1.3 | | | | |
| M | 50 | Iso | CD | 9:01 | 1.4 | 21 | 1.1 | 1.1 | 1.5 | 1.6 | 0.9 | 0.9 |
| M | 50 | BSO | — | — | 1.4 | 13 | 1.2 | 1.2 | 2.9 | 3.6 | 0.7 | 0.7 |
| M | 50 | BSO | MCT | 3:01 | 1.3 | 12 | 1.4 | 1.6 | 1.6 | 1.8 | 1.1 | 1.4 |
| M | 50 | BSO | CD | 3.4:1 | 1.6 | 18 | 1.4 | 1.3 | 1.8 | 1.8 | 1.2 | 1.2 |

TABLE 3
DHA, HMF and MGO content of honey/cannabidiol/emulsifier (carrier) compositions.

| | Mix | CBD | | Carrier:CBD | CBD in | | Composition (mg/kg) | | |
|---|---|---|---|---|---|---|---|---|---|
| Honey | Temp | source | Carrier | source wt:wt | honey mg/g | Fraction | DHA | HMF | MGO |
| M | 50 | BSO | Lec | 10:01 | 1.4 | 1 | 290 | 42 | 262 |
| M | 60 | BSO | Lec | 10:01 | 1.4 | 4 | 284 | 42.4 | 245 |
| M | 70 | BSO | Lec | 10:01 | 1.4 | 5 | 292 | 45.4 | 239 |
| M | 50 | BSO | Lec | 10:01 | 7 | 2 | 250 | 33 | 181 |
| C | 50 | BSO | Lec | 10:01 | 1.4 | 3 | | | |
| M | 50 | BSO | Lec | 5:01 | 1.4 | 6 | | | |
| M | 50 | BSO | Lec | 2:01 | 1.4 | 7 | | | |
| M | 50 | Iso | Lec | 10:01 | 1.4 | 8 | | | |
| M | 50 | Iso | Lec | 10:01 | 7 | 9 | 280 | 40 | 250 |
| M | 50 | BSO | CD | 9:01 | 1.4 | 14 | 290 | 42 | 267 |
| M | 60 | BSO | CD | 9:01 | 1.4 | 19 | 293 | 38.5 | 272 |
| M | 70 | BSO | CD | 9:01 | 1.4 | 20 | 289 | 40.7 | 264 |
| C | 50 | BSO | CD | 9:01 | 1.4 | 17 | | | |
| M | 50 | Iso | CD | 9:01 | 1.4 | 21 | 290 | 42 | 269 |
| M | 50 | BSO | — | — | 1.4 | 13 | 290 | 43 | 276 |
| M | 50 | BSO | MCT | 3:01 | 1.3 | 12 | 290 | 43 | 277 |
| M | 50 | BSO | CD | 3.4:1 | 1.6 | 18 | 300 | 44 | 277 |
| M+ | | | | | | | 275 | 44.6 | 275 |

M+ = A sample of supplied Mānuka honey.

### Example 3

Four different CBD-in-honey formulations were prepared in this Example (Table 4), both with cannabidiol isolate and broad spectrum distillate (BSD) and with two different γ-CD loadings, following the method described in previous work. In all cases, γ-CD was mixed with water at a 1:1 mass ratio to produce a paste. The cannabidiol isolate or BSD was dissolved in isopropyl alcohol (IPA) and added to the γ-CD paste under continuous stirring. Note that the 1:4 or 1:9 ratios refer to CBD: γ-CD, so the amount of BSD used was calculated to achieve that ratio of CBD based on a 72% CBD content in the BSD as reported by the manufacturer, while CBD isolate was assumed to contain 100% CBD. Additional water was added under continuous stirring to dilute IPA to lower levels for freeze drying, resulting in a low viscosity aqueous suspension. This solution was frozen to −80° C., then freeze-dried. Weights given in Table 5 are the material recovered.

TABLE 4
Formulations prepared in Example 3.

| | Broad | | |
|---|---|---|---|
| | Spectrum | | Blank |
| | Distillate | Isolate | formulations¹ |
| 1:4 CBD:γ-CD | Formulation 1 | Formulation 3 | Formulation 5 |
| 1:9 CBD:γ-CD | Formulation 2 | Formulation 4 | Formulation 6 |

¹Contain no CBD with γ-CD present at the equivalent ratio.

TABLE 5
CBD-containing formulations prepared in Example 3.

| | Formulation | Formulation | Formulation | Formulation |
|---|---|---|---|---|
| | 1 | 2 | 3 | 4 |
| γ-CD (g) | 8.00 | 18.00 | 8.00 | 18.01 |
| Water (g) | 8.01 | 18.03 | 8.02 | 18.02 |
| CBD source² | 2.71 | 2.83 | 2.00 | 2.00 |
| (g) | | | | |
| IPA (g) | 8.34 | 8.28 | 6.03 | 6.03 |
| Additional | ~80 | ~80 | ~80 | ~80 |
| water (g) | | | | |
| Final (dry) | 10.2 | 20.0 | 5.7 | 16.8 |
| complex | | | | |
| obtained (g) | | | | |
| Complex used | 3.55 | 7.09 | 3.55 | 7.10 |
| in honey | | | | |
| mixture (g) | | | | |
| Honey (g) | 96.43 | 92.89 | 96.53 | 92.95 |

²BSD for Formulations 1 and 2, cannabidiol isolate for Formulations 3 and 4.

The resulting dried powder was then finely ground and added to honey that had been prewarmed to approx. 50° C. under vigorous stirring.

Two additional non-CBD honey formulations (Formulation 5 and 6) were prepared by blending γ-CD into honey at ratios equivalent to those above.

Small samples of Formulations 1-4 were centrifuged at 2400 rpm for 10 minutes after allowing them to cool slightly. Samples (1-2 g) were then taken from the top and bottom of the tube for cannabinoid analysis. In some samples a white foamy layer appeared on the surface after centrifuging. This is thought to be associated with release of air from the honey during centrifugation.

CBD Concentration

The target CBD concentration in the final honey product was 50 mg CBD per teaspoon of honey product. Assuming 1 teaspoon of honey=7 g, this is equivalent to 7.1 mg CBD per g of honey. The calculations carried out for each of the formulations were based on this target final concentration and the specified concentration of CBD in the full spectrum distillate (72%). Note that analysis revealed a CBD concentration of 77% in the supplied distillate (Table 5).

The measured cannabinoid concentrations in the final products are reported in Table 7. All honey formulations are generally within range of the target concentration of 7.1 mg CBD/g (i.e., 0.71%). The content of CBD and CBN (cannabinol) in the γ-CD complexes are consistent with the expected amounts based on the CBD and CBN concentrations in the supplied BSD and isolate.

TABLE 6
Cannabinoid concentration (%) of full
spectrum distillate (BSD).

| CBD | CBN | THCA | CBDA | THC |
|---|---|---|---|---|
| 77 | 1.8 | <0.4 | <0.5 | <0.5 |

CBD: cannabidiol; CBN: cannabinol; THCA: tetrahydrocannabinolic acid; CBDA: Cannabidiolic acid; THC: tetrahydrocannabinol.

TABLE 7
Cannabinoid concentrations (%) of final formulations. CBD: cannabidiol; CBN: cannabinol;

| Sample | Description | CBD | CBN | THCA | CBDA | THC |
|---|---|---|---|---|---|---|
| Complex 1 | Cyclodextrin | 19 | 0.46 | <0.1 | <0.1 | <0.1 |
| | complex | | | | | |
| | 1:4 CBD: γ-CD | | | | | |
| | (distillate) | | | | | |
| Complex 2 | Cyclodextrin | 10 | 0.25 | <0.06 | <0.07 | <0.07 |
| | complex | | | | | |
| | 1:9 CBD: γ- | | | | | |
| | CD (distillate) | | | | | |
| Complex 3 | Cyclodextrin | 21 | <0.1 | <0.1 | <0.1 | <0.1 |
| | complex | | | | | |
| | 1:4 CBD: γ- | | | | | |
| | CD (isolate) | | | | | |
| Complex 4 | Cyclodextrin | 9.7 | <0.06 | <0.06 | <0.06 | <0.06 |
| | complex | | | | | |
| | 1:9 CBD: γ- | | | | | |
| | CD (isolate) | | | | | |
| Formulation 1 | Honey | 0.70 | 0.017 | <0.004 | <0.005 | <0.005 |
| | formulation | | | | | |
| | 1:4 CBD: γ- | | | | | |
| | CD (distillate) | | | | | |
| Formulation 1 | Post | 0.91 | 0.022 | <0.004 | <0.004 | <0.004 |
| top | centrifugation, | | | | | |
| | top phase | | | | | |
| | 1:4 CBD: γ- | | | | | |
| | CD (distillate) | | | | | |
| Formulation 1 | Post | 0.62 | 0.015 | <0.004 | <0.004 | <0.004 |
| bottom | centrifugation, | | | | | |
| | bottom phase | | | | | |
| | 1:4 CBD: γ- | | | | | |
| | CD (distillate) | | | | | |
| Formulation 2 | Honey | 0.76 | 0.019 | <0.004 | <0.005 | <0.004 |
| | formulation | | | | | |
| | 1:9 CBD: γ- | | | | | |
| | CD (distillate) | | | | | |
| Formulation 2 | Post | 0.74 | 0.019 | <0.005 | <0.005 | <0.005 |
| top | centrifugation, | | | | | |
| | top phase | | | | | |
| | 1:9 CBD: γ- | | | | | |
| | CD (distillate) | | | | | |
| Formulation 2 | Post | 0.76 | 0.019 | <0.004 | <0.004 | <0.004 |
| bottom | centrifugation, | | | | | |
| | bottom phase | | | | | |
| | 1:9 CBD: γ- | | | | | |
| | CD (distillate) | | | | | |
| Formulation 3 | Honey | 0.66 | <0.005 | <0.005 | <0.005 | <0.005 |
| | formulation | | | | | |
| | 1:4 CBD: γ- | | | | | |
| | CD (isolate) | | | | | |
| Formulation 3 | Post | 0.73 | <0.005 | <0.005 | <0.005 | <0.005 |
| top | centrifugation, | | | | | |
| | top phase | | | | | |
| | 1:4 CBD: γ- | | | | | |
| | CD (isolate) | | | | | |
| Formulation 3 | Post | 0.64 | <0.005 | <0.005 | <0.005 | <0.005 |
| bottom | centrifugation, | | | | | |
| | bottom phase | | | | | |
| | 1:4 CBD: γ- | | | | | |
| | CD (isolate) | | | | | |
| Formulation 4 | Honey | 0.70 | <0.004 | <0.004 | <0.004 | <0.004 |
| | formulation | | | | | |
| | 1:9 CBD: γ- | | | | | |
| | CD (isolate) | | | | | |
| Formulation 4 | Post | 0.69 | <0.004 | <0.004 | <0.005 | <0.005 |
| top | centrifugation, | | | | | |
| | top phase | | | | | |
| | 1:9 CBD: γ- | | | | | |
| | CD (isolate) | | | | | |
| Formulation 4 | Post | 0.70 | <0.005 | <0.004 | <0.005 | <0.005 |
| bottom | centrifugation, | | | | | |
| | bottom phase | | | | | |
| | 1:9 CBD: γ- | | | | | |
| | CD (isolate) | | | | | |

THCA: tetrahydrocannabinolic acid; CBDA: Cannabidiolic acid; THC: tetrahydrocannabinol.

Physical Stability

Samples of the formulations were centrifuged at 2400 rpm for 10 minutes while still slightly warm, and subsamples from the top and bottom of the tube were collected separately for CBD analysis to determine if segregation had occurred.

As in previous Examples 1 and 2 (above), a foamy layer at the top of all γ-CD formulations was observed after centrifuging; this is believed to be associated with deaeration of the honey rather than separation of components from within the honey. It is however possible the deaeration could itself cause some phase separation as the air rises through the honey, and so care should be taken during mixing of the formulations that excess levels of air are not introduced by the mixing process or that a controlled deaeration process is carried out before fully homogenising the products. A slightly higher concentration of CBD was measured in the top samples post-centrifugation (Table 7) for formulations 1 and 3. These formulations had a lower γ-CD loading, possibly leading to poor stability and separation of CBD during centrifugation.

Honey Attributes

As observed in previous Examples (Examples 1 and 2), addition of CBD and complexing agents to make the trial formulations did not appear to have any detrimental effect on the properties of honey, and there is no substantial difference between the starting honey and the two formulations due to either the processing or the short storage time between formulation and analysis. Physiologically, MGO is regarded as a ‘Reactive Oxygen Species’ i.e., an oxidizing agent. These results suggest that there are no oxidative reactions occurring during the formulation process because there is no apparent decrease in MGO, particularly for the CBD distillate samples. It is worth noting that formulations 1, 3 and 5 contained 96% honey, whereas formulations 2, 4 and 6 had a large amount of γ-CD present and therefore only 93% honey. In order to more accurately compare the results for pure honey with those for the different formulations, they need to be standardised to 100% honey. Both sets of values are shown in Table 8.

TABLE 8
Honey properties (mg/kg)

| | | Mānuka | Form. | Form. | Form. | Form. | Form. | Form. |
|---|---|---|---|---|---|---|---|---|
| | | honey | 1 | 2 | 3 | 4 | 5 | 6 |
| As produced | DHA | 471 | 461 | 463 | 448 | 433 | 458 | 446 |
| | HMF | 16.7 | 12.8 | 16.2 | 10.6 | 10.2 | 16.4 | 13.7 |
| | MGO | 307 | 300 | 305 | 290 | 283 | 300 | 291 |
| Standardised to 100% | DHA | 471 | 478 | 498 | 464 | 466 | 475 | 480 |
| honey | HMF | 16.7 | 13.3 | 17.4 | 11 | 11 | 17 | 14.7 |
| | MGO | 307 | 311 | 328 | 301 | 305 | 311 | 313 |

### Example 4

A cannabigerol (CBG)-in-honey formulation was prepared using CBG isolate with a γ-CD:CBG loading of 9:1, following the method described in the previous Examples.

9 g of γ-CD was mixed with water at a 1:1 mass ratio to produce a paste. CBG isolate (1 g) was dissolved in isopropyl alcohol (IPA, 3 g) and added to the γ-CD paste under continuous stirring. Additional water (˜50 g) was added under continuous stirring to dilute IPA to lower levels for freeze drying, resulting in a low viscosity aqueous suspension. This solution was frozen to −80° C., then freeze-dried. The resulting dried powder was then finely ground and added to honey that had been prewarmed to approx. 50° C. under vigorous stirring. Details of the composition of honey and CBG are shown in Table 9 below:

TABLE 9
CBG-containing formulations prepared in this Example.

| | Formulation 1 |
|---|---|
| γ-CD (g) | 9 |
| Water (g) | 9 |
| CBG (g) | 1 |
| IPA (g) | 3 |
| Additional water (g) | ~50 |
| Final (dry) complex obtained (g) | 9.2 |
| Complex used in honey mixture (g) | 1.4 |
| Honey (g) | 98.6 |

A sample of the product was centrifuged at 2400 rpm for 10 minutes while still relatively warm. Subsamples (1-2 g) were then taken from the top and bottom of the tube for CBG analysis. A white foamy layer appeared on the top surface after centrifuging. This is thought to be associated with release of air from the honey during centrifugation and consistent with observations in previous Examples.

The target CBG concentration in the final honey product was 1.4 mg CBG per g of honey. The measured CBG concentration in the final product is shown in Table 10.

The level of CBG in the γ-CD complex (104 mg per g) is consistent with the expected value of 10%. While the CBG levels in the final product (1.2 mg per g) is slightly below the target of 1.4 mg CBG per g, this is probably within the expected variance of the current analytical method and therefore not a significant departure from expectations. The reported results are based on the mass spectral data; however, the results were also confirmed by reviewing the UV data recorded at 275 nm, which was found to be consistent with the results reported below.

A small sample of the final formulation was centrifuged at 2400 rpm for 10 minutes while still slightly warm, and subsamples from the top and bottom of the tube were collected separately for CBG analysis to determine if segregation had occurred.

Physical Stability

As in previous Examples 1-3, a foamy layer at the top of the formulation was observed after centrifuging. This is believed to be associated with deaeration of the honey rather than separation of components from within the honey per se. Analysis of the top and bottom phase post centrifugation of the final product did not show any significant segregation of CBG (see Table 10).

TABLE 10
CBG content of different fractions

| | | | Top phase | Bottom phase |
|---|---|---|---|---|
| | CBG-γCD | Final CBG | post | post |
| | complex | formulation | centrifugation | centrifugation |
| CBG | 104 | 1.2 | 1.3 | 1.2 |
| (mg/g) | | | | |

Honey Attributes

As observed in Examples 1-3, addition of CBG and γ-CD to make the trial formulation did not appear to have any detrimental effect on the properties of honey, and there is no substantial difference between the starting honey and the final formulation (Table 11). The results for the neat honey are consistent with the results obtained for the same honey in Example 3, indicating good stability during storage.

TABLE 11
Honey properties (mg/kg)

| | Mānuka | CBG |
|---|---|---|
| | honey | Formulation |
| DHA | 465 | 486 |
| HMF | 14.4 | 15.9 |
| MGO | 316 | 310 |

### Example 5

Stability trials for CBD-infused Mānuka honey formulations were performed. Two different formulations were prepared using either lecithin or gamma cyclodextrin in the production of a CBD-emulsifier combination, and four different types of packaging material were used.

In an accelerated aging study, the formulations were stored in a controlled environment (40° C., 75% relative humidity) over 6 months, with samples tested at 0, 3 and 6 months for CBD content and/or selected quality markers in the honey (DHA, HMF and MGO). In the room temperature study, samples were stored in a controlled environment at 25° C., 60% relative humidity.

Lecithin formulations were found to have low stability at 40° C., with a ˜70% loss of CBD. There was also an ˜86% loss of MGO after 6 months at this storage temperature and a corresponding increase of HMF to values above 250 mg/g. γ-CD formulations, on the other hand, showed very good stability at 40° C. with only −14% loss of CBD.

As expected, the changes in levels of tested compounds with storage time at room temperature was much less than at 40° C.

Lecithin Formulation

Sunflower lecithin (27.02 g) was mixed with CBD isolate (3.01 g) at 45° C. until homogeneously blended to produce a mixture having a 1:9 CBD:lecithin ratio. A fraction of this mixture (22.7 g) was added to Mānuka honey (1477 g) at 60° C. for 1 hour with stirring. The resulting mixture was then transferred to the supplied storage containers and labelled as “Formulation 3” (F3).

γ-Cyclodextrin Formulation

γ-CD (36.05 g) was mixed with water (36.48 g) at a 1:1 mass ratio to produce a uniform paste. CBD isolate (4.01 g) was dissolved in isopropyl alcohol (12.05 g) and added to the γ-CD paste with continuous stirring to produce a 1:9 CBD:γ-CD (w/w) ratio. Additional water (˜380 mL) was added with continuous stirring to ensure the final mixture, resulting in a low viscosity aqueous suspension. IPA was removed by evaporation under vacuum at 40° C., and the resulting solution was frozen to −80° C., then freeze-dried. A fraction of the resulting dry complex (20.8 g) was then finely ground and mixed with Mānuka honey (1479 g) at 60° C. for 1 hour with stirring. The resulting mixture was then transferred to the supplied storage containers and labelled as “Formulation 4” (F4).

Accelerated Aging Study

The results of the accelerated study are shown in Table 12. All formulations are generally within range of a target initial concentration of 1.4 mg CBD/g. After three months, there is a sharp decrease (˜50%) in CBD concentration for all lecithin formulation samples (F3) at 40° C. storage temperature, regardless of container type. This loss of CBD continues over time and reaches ˜70% after six months. In contrast, the γ-CD formulation samples (F4) showed excellent stability over three months, and only a small loss of CBD (˜14%) after six months for all container types.

Cannabidiol (CBD), DHA, HMF and MGO average values measured over six months for the two different formulations, as well as the control (pure honey), are shown in Table 12. The results show significant degradation of the honey after 6 months at 40° C.

TABLE 12
Evolution of CBD and honey properties in different formulations over time at 40° C.
75% RH.

| | | | | CBD | DHA | HMF | MGO |
|---|---|---|---|---|---|---|---|
| Sample | Formulation | Container | Month | (mg/g) | (mg/g) | (mg/g) | (mg/g) |
| F3-PET-40 T = 0 | Isolate + | PET | 0 | 1.5 | 470 | 15.4 | 291 |
| F3-PET-40 T = 3 | Lecithin | | 3 | 0.7 | — | — | — |
| F3-PET-40 T = 6 | | | 6 | 0.4 | 12 | >259 | 39 |
| F3-PP-40 T = 0 | Isolate + | PP | 0 | 1.4 | 477 | 15.7 | 293 |
| F3-PP-40 T = 3 | Lecithin | | 3 | 0.7 | — | — | — |
| F3-PP-40 T = 6 | | | 6 | 0.4 | <12 | >263 | 40 |
| F3-GLASS-40 | Isolate + | GLASS | 0 | 1.4 | 466 | 15.2 | 291 |
| T = 0 | Lecithin | | | | | | |
| F3-GLASS-40 | | | 3 | 0.7 | — | — | — |
| T = 3 | | | | | | | |
| F3-GLASS-40 | | | 6 | 0.4 | <12 | >250 | 40 |
| T = 6 | | | | | | | |
| F4-PET-40 T = 0 | Isolate + γ- | PET | 0 | 1.5 | 472 | 13 | 298 |
| F4-PET-40 T = 3 | CD | | 3 | 1.4 | — | — | — |
| F4-PET-40 T = 6 | | | 6 | 1.3 | 15 | >232 | 73 |
| F4-PP-40 T = 0 | Isolate + γ- | PP | 0 | 1.4 | 471 | 12.7 | 296 |
| F4-PP-40 T = 3 | CD | | 3 | 1.3 | — | — | — |
| F4-PP-40 T = 6 | | | 6 | 1.2 | <11 | >226 | 64 |
| F4-GLASS-40 | Isolate + γ- | GLASS | 0 | 1.4 | 473 | 12.8 | 298 |
| T = 0 | CD | | | | | | |
| F4-GLASS-40 | | | 3 | 1.4 | — | — | — |
| T = 3 | | | | | | | |
| F4-GLASS-40 | | | 6 | 1.2 | 15 | >221 | 75 |
| T = 6 | | | | | | | |
| Control-40 | control | GLASS | 0 | | 473 | 13.6 | 303 |
| T = 0 | (honey) | | | | | | |
| Control-40 | | | 6 | — | 11 | >234 | 92 |
| T = 6 | | | | | | | |

Room Temperature Study

The measured CBD concentration in each sample after storage for 6 months at 25° C. 60% RH is shown in Table 13. As observed in the accelerated study, lecithin samples showed moderate loss of CBD after 6 months (14-27% loss). Samples in PET containers, followed by PP, appear to have the poorest stability, whereas glass and HDPE showed similar results. γ-CD samples showed excellent stability, with no apparent loss of CBD after 6 months.

In terms of honey properties, the control sample (pure honey) showed a moderate decrease in DHA but only a small decrease of MGO. The results for the γ-CD sample stored in a glass container are generally consistent with the control sample, and indicate that CBD-γCD complexes do not appear to have a negative effect on the stability of honey. In contrast, the results for the lecithin samples show a significant decrease in MGO over time compared to control and γ-CD samples.

TABLE 13
Evolution of CBD and honey properties in different formulations over time
at 25° C. 60% RH.

| | | | | CBD | DHA | HMF | MGO |
|---|---|---|---|---|---|---|---|
| Sample | Formulation | Container | Month | (mg/g) | (mg/g) | (mg/g) | (mg/g) |
| F3-PET-25 | Isolate + | PET | 0 | 1.5 | 470 | 15.4 | 291 |
| T = 0 | Lecithin | | | | | | |
| F3-PET-25 | | | 6 | 1.1 | — | — | — |
| T = 6 | | | | | | | |
| F3-PP-25 T = 0 | Isolate + | PP | 0 | 1.4 | 477 | 15.7 | 293 |
| F3-PP-25 T = 6 | Lecithin | | 6 | 1.1 | — | — | — |
| F3-GLASS-25 | Isolate + | GLASS | 0 | 1.4 | 466 | 15.2 | 291 |
| T = 0 | Lecithin | | | | | | |
| F3-GLASS-25 | | | 6 | 1.2 | — | — | — |
| T = 6 | | | | | | | |
| F3-HDPE-25 | Isolate + | HDPE | 0 | 1.3 | 468 | 15.1 | 289 |
| T = 0 | Lecithin | | | | | | |
| F3-HDPE-25 | | | 6 | 1.1 | — | — | — |
| T = 6 | | | | | | | |
| F4-PET-25 | Isolate + | PET | 0 | 1.5 | 472 | 13 | 298 |
| T = 0 | γ-CD | | | | | | |
| F4-PET-25 | | | 6 | 1.4 | — | — | — |
| T = 6 | | | | | | | |
| F4-PP-25 T = 0 | Isolate + | PP | 0 | 1.4 | 471 | 12.7 | 296 |
| F4-PP-25 T = 6 | γ-CD | | 6 | 1.4 | — | — | — |
| F4-GLASS-25 | Isolate + | GLASS | 0 | 1.4 | 473 | 12.8 | 298 |
| T = 0 | γ-CD | | | | | | |
| F4-GLASS-25 | | | 6 | 1.5 | 279 | 24.7 | 292 |
| T = 6 | | | | | | | |
| F4-HDPE-25 | Isolate + | HDPE | 0 | 1.4 | 467 | 12.8 | 299 |
| T = 0 | γ-CD | | | | | | |
| F4-HDPE-25 | | | 6 | 1.4 | — | — | — |
| T = 6 | | | | | | | |
| Control-25 | control | GLASS | 0 | — | 473 | 13.6 | 303 |
| T = 0 | (honey) | | | | | | |
| Control-25 | | | 6 | — | 286 | 29 | 278 |
| T = 6 | | | | | | | |

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

Unless specifically defined otherwise, all technical and scientific terms used herein shall be taken to have the same meaning as commonly understood by one of ordinary skill in the art.

It is intended that reference to a range of numbers disclosed herein (e.g. 1 to 10) also incorporates reference to all related numbers within that range (e.g. 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

The term “and/or”, e.g., “X and/or Y” shall be understood to mean either “X and Y” or “X or Y” and shall be taken to provide explicit support for both meanings or for either meaning.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification. The specific compositions and methods described herein are representative of preferred examples and are exemplary and not intended as limitations on the scope of the invention. Other aspects and examples will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed as essential. Thus, for example, in each instance described or used herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The assays and methods illustratively described herein suitably may be practiced in differing orders of steps, and that they are not necessarily restricted to the orders of steps indicated herein or in the claims. Further, as used or described herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts disclosed herein may be resorted to by those skilled in the art, and that such modifications and variations are considered to be within the scope of this invention as described herein, and as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

1. A composition comprising:
a honey;
a cannabinoid; and
an emulsifier.

2. The composition of claim 1, wherein the honey comprises Mānuka honey.

3. The composition of claim 1 or 2, wherein the honey comprises methylglyoxal.

4. The composition of any one of the preceding claims, wherein the water content of the composition is less than 20%.

5. The composition of any one of the preceding claims, wherein the cannabinoid is selected from the group consisting of tetrahydrocannabinol (THC), cannabidiol (CBD), cannabigerol (CBG), cannabinol (CBN), tetrahydrocannabinolic acid (THCA), cannabidiolic acid (CBDA), and tetrahydrocannabivarin.

6. The composition of any one of the preceding claims, comprising at least 1 mg cannabinoid per gram of composition.

7. The composition of any one of the preceding claims, comprising at least 1 mg cannabinoid per gram of composition at 6 months after preparation, or at 12 months after preparation, or at 18 months after preparation, or at 24 months after preparation.

8. The composition of any one of the preceding claims, comprising at least 250 mg methylglyoxal per kilogram of composition.

9. The composition of any one of the preceding claims, comprising at least 250 mg methylglyoxal per kilogram of composition at 6 months after preparation, or at 12 months after preparation, or at 18 months after preparation, or at 24 months after preparation.

10. The composition of any one of the preceding claims, wherein the emulsifier comprises cyclodextrin.

11. The composition of claim 10, comprising a cyclodextrin-cannabinoid complex.

12. The composition of claim 10 or 11, wherein the ratio of cyclodextrin to cannabinoid is greater than 4:1 (w/w).

13. The composition of any one of claims 10-12, wherein the ratio of cyclodextrin to cannabinoid is about 9:1 (w/w).

14. The composition of any one of the preceding claims, comprising at least 90% w/w honey.

15. The composition of any one of the preceding claims, wherein the cannabinoid is uniformly distributed in the composition, preferably at 6 months after preparation, or at 12 months after preparation, or at 18 months after preparation, or at 24 months after preparation.

16. A method for preparing the composition of any one of claims 1 to 15, comprising the following steps:
a) Combining the cannabinoid and the emulsifier to form a cannabinoid-emulsifier combination; and
b) Combining the combination with honey.

17. The method of claim 16, wherein the emulsifier comprises cyclodextrin.

18. The method of claim 17, wherein the cyclodextrin and cannabinoid are combined in the presence of water and a water-miscible solvent.

19. The method of claim 18, wherein the solvent comprises isopropyl alcohol.

20. The method of any one of claims 16-19, wherein the cannabinoid-emulsifier combination is dried prior to step b.

21. The method of any one of claims 16-20, wherein the cannabinoid-emulsifier combination is provided as a powder prior to step b.

22. A composition comprising:
Methylglyoxal
A cannabinoid; and
An emulsifier.

23. The composition of claim 22, wherein the emulsifier is cyclodextrin.

24. The composition of claim 22 or 23, further comprising honey.
